(19) United States
(12) Patent Application Publication
(10) Pub. No.: US 2004/0153045 A1
(43) Pub. Date: Aug. 5, 2004

(54) Infant wearing apparel with a detachable fastener

(76) Inventor: Luk Baskerville, Calgary (CA)

Correspondence Address: David S. Thompson, South 7 Howard, # 418, Spokane, WA, 99201, US

(21) Appl. No.: 09/972,561

(22) Filed: Oct. 9, 2001

Related U.S. Application Data
(60) Provisional application No. 60/265,686, filed on Jan. 31, 2001.

(51) Int. Cl. A61F 13/15

(52) U.S. Cl. 604/391

(57) ABSTRACT

An infant wearing apparel (10) is usable as a swimsuit, training pants, reusable diaper or article of clothing for any purpose. A preferred version of the infant apparel includes a body made of sheet material (20) having an absorbent inside surface and a waterproof outside surface. Hook and loop type fasteners are used to secure the garment. Left and right loop fasteners (40) are carried on an inside upper surface of the rear of the body made of sheet material. A front loop fastener strip (60) is carried on an outside upper surface of the front of the body made of sheet material. Left and right fabric extensions (80) include hook connectors on first and second sides and connect the left and right loop fasteners to the left and right ends of the front loop fastener strip, respectively.

### CROSS-REFERENCES

This application is a continuation in part of a provisional application filed by the same inventor on Jan. 31, 2001, having Ser. No. 60/265,686.

### BACKGROUND

The use of hook and loop fasteners, typically known by the trademark VELCRO, is widespread. However, application of hook and loop fasteners for use in the area of infant's reusable diapers and swim wear has been less than successful. There are a number of reasons for this problem. First, during the laundering process, the hook portion of the fastener typically adheres to other items in the washing machine. This can cause damage to many items in a short period of time. Additionally, during the drying process, lint and foreign material can adhere to the hook portion of the fastener, reducing its effectiveness. More importantly, in many applications, use of such fasteners results in irritation to the skin of the infant.

Accordingly, there is a need for a new structure using hook and loop fasteners that is adapted for use with infants reusable clothing, diapers and swim wear.

### SUMMARY

The present invention is directed to an apparatus that satisfies the above needs. Novel infant wearing apparel is disclosed that includes a detachable fastener. The fastener includes fabric extensions that may be used to adjust the size of the diaper, while preventing irritation to the skin and improving the laundering process.

The infant wearing apparel with detachable hook and loop fasteners of the present invention provides some or all of the following structures.

(A) A body made of sheet material includes a front portion, a back portion, and leg notches.

(B) Left and right loop fasteners are attached to left and right inside upper corner surfaces of the back portion.

(C) A front loop fastener strip is carried by the outer upper edge of the front portion of the body made of sheet material.

(D) Left and right fastener extensions connect left and right ends of the front loop fastener strip to the left and right loop fasteners, respectively. Each fastener extension has hook fasteners on a first side at a first end and on a second side at a second end. Smooth surfaces are on sides opposite the hook fasteners.

(E) Optionally, each fastener extension includes an auxiliary fastener, attached to a middle portion of the fastener extension. Each auxiliary fastener includes a first surface having hook fasteners and a smooth second surface. The hook portion engages left and right outside rear fastening patches. Accordingly, the connection between the left and right corners of the upper rear edge and the left and right sides of the front fastening strip is more secure.

### DRAWINGS

These and other features, aspects, and advantages of the present invention will become better understood with regard to the following description, appended claims, and accompanying drawings where:

FIG. 1 shows a perspective view of an implementation of the infant wearing apparel with detachable hook and loop fasteners.

FIG. 2 shows a planar view of the outside surface of the apparel of FIG. 1.

FIG. 3 shows a planar view of the inside surface of the apparel of FIG. 1.

FIGS. 4A, 4B and 4C show front, side and back views of an exemplary fastener extension.

FIG. 5 is a view similar to that of FIG. 2, showing a second version of the infant wearing apparel.

FIG. 6 shows front, side and back views of a second version of the fastener extension associated with the version of FIG. 5.

### DESCRIPTION

An infant wearing apparel 10 is usable as a swimsuit, training pants, reusable diaper or article of clothing for any purpose. A preferred version of the infant apparel includes a body made of sheet material 20 having an absorbent inside surface and a waterproof outside surface. Hook and loop type fasteners are used to secure the garment. Left and right loop fasteners 40 are carried on an inside upper surface of the rear of the body made of sheet material. A front loop fastener strip 60 is carried on an outside upper surface of the front of the body made of sheet material. Left and right fabric extensions 80 include hook connectors on first and second sides and connect the left and right loop fasteners to the left and right ends of the front loop fastener strip, respectively. Optionally, the rear of the body made of sheet material may include outside loop fasteners 110 located on the outside upper surface of the rear of the body made of sheet material, and each fabric extension may include an auxiliary fastener 100. By connecting the outside loop fasteners to the hook-type auxiliary fastener of the fabric extension, a more secure connection is made.

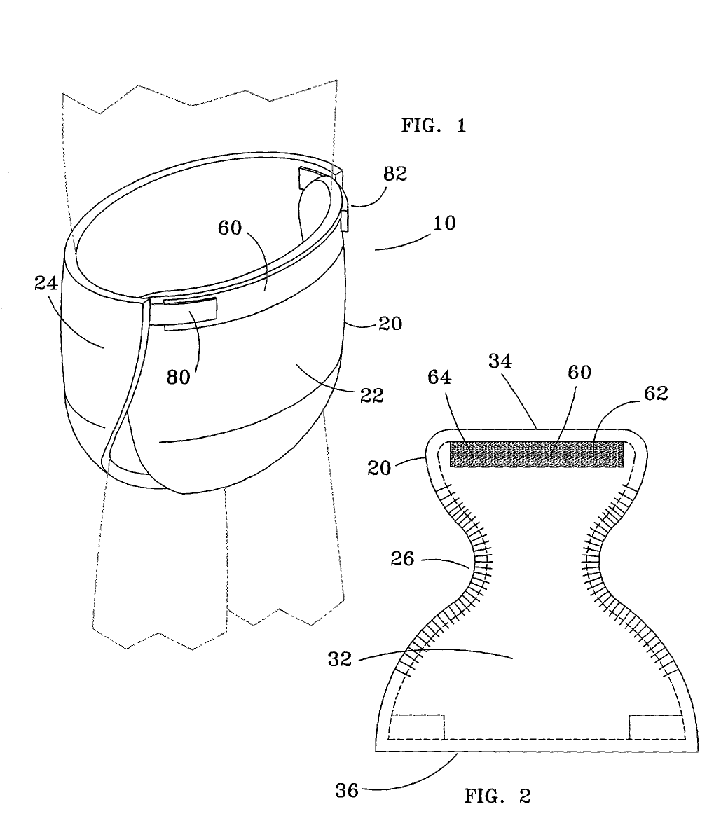

Referring to FIGS. 1, 2 and 3, a body made of sheet material 20 includes a front portion 22, a back portion 24, and leg notches 36. The leg notches may have an elastic element to provide a secure fit around the infant's legs. An inside surface 28 is typically made of absorbent material. An extra absorbent layer 30 may be added, as needed. An outside surface 32 is typically made of plastic or other waterproof material. An upper front edge 34 and an upper rear edge 36 are oriented upwardly during wear.

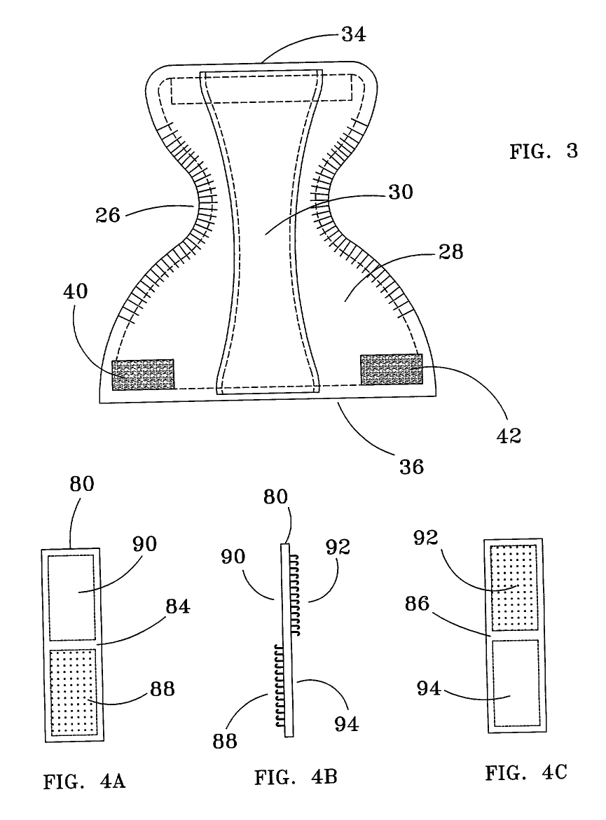

Referring to FIG. 3, left and right loop fasteners 40, 42 are attached to the inside surface of the upper left and right corners of the upper rear edge 36 of the back portion 24 of the body made of sheet material 20, respectively. The loop fasteners, unlike hook fasteners, are soft. Therefore, if and when they come into contact with a baby's skin, they are not going to be a cause of irritation. A preferred loop fastener is the soft portion of a VELCRO fastener, although similar fasteners may be substituted.

Referring to FIGS. 1 and 2, a front loop fastener strip 60 is carried adjacent to the outer upper edge 34 of the front portion 22 of the body 20 made of sheet material. A preferred version of the loop fastener is also made of the soft portion of a VELCRO fastener, although similar fasteners could be substituted. As will be seen, a left end 62 of the fastener strip is releasably attachable to a left fastener extension 80, while a right end 64 is releaseably attachable to a right fastener extension 82.

Referring to FIGS. 1 and 4, left and right fastener extensions 80, 82 connect left and right ends 62, 64 of the front loop fastener strip 60 to the left and right loop fasteners, 40, 42 respectively. Each fastener extension includes first and second sides 84, 86. The first side 84 of each fastener extension includes a first hook surface 88 and a first smooth surface 90. The second side 86 of each fastener extension includes a second hook surface 92 and a second smooth surface 94.

As seen in FIG. 1, in use, the left fastener extension 80 attaches the left loop fastener 40 to the left end 62 of the front fastening strip 60. In particular, the first hook surface 88 of is attached to the left loop fastener 40. The second hook surface 92 is attached to the left end 62 of the front loop-fastening strip 60. Similarly, the right fastener extension 82 attaches the right loop fastener 42 to the right end 64 of the front fastening strip 60.

Optionally, the left and right fastener extensions 80, 82 may each include an auxiliary fastener 100. As seen in FIG. 6, the auxiliary fastener 100 is attached to a middle portion 106 of the fastener extension. Each auxiliary fastener includes a first surface 102 having hook fasteners and a second surface 104 that is smooth.

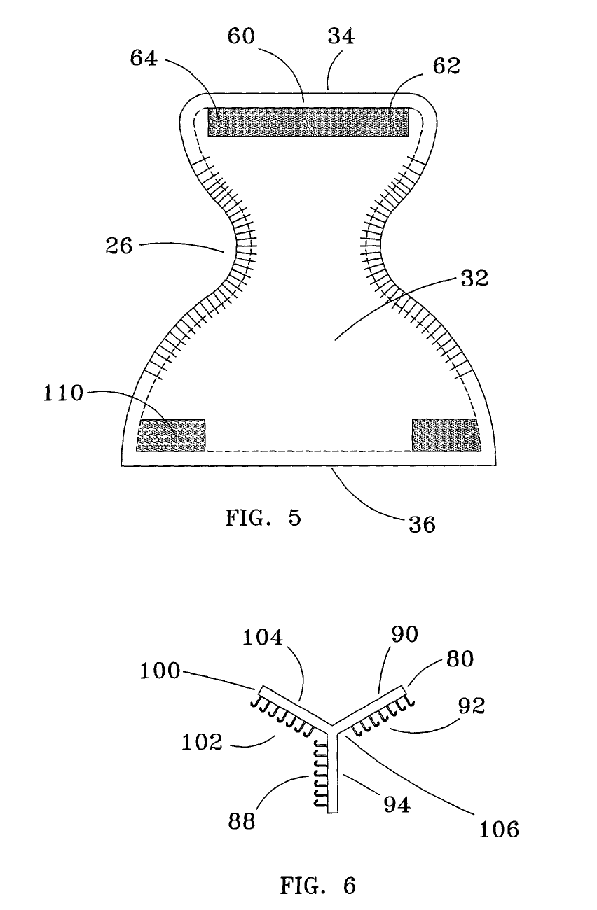

As seen in FIG. 5, in an optional embodiment associated with the auxiliary fastener extensions 80, 82, left and right outside rear fastening patches 110, 112 are carried by the outside surface of the back portion 24 of the body made of sheet material 20. Accordingly, the left and right outside rear fastening patches 110, 112 are on the outside surface 32 of the body 20 made of sheet material in the same general location wherein the left and right loop fasteners 40, 42 are positioned on the inside of the body made of sheet material.

In operation, the hook surface 102 is attached to the outside rear fastener patch 110, on both the left and right sides of the garment 10. As a result, the loop fasteners 40 and 110 (e.g. the “soft” part of a VELCRO(R) fastener) are attached to the hook surfaces 40, 110 (e.g. the “prickly” plastic part of a VELCRO(R) fastener). Thus, a better connection is made between the fastener extensions and the left and right ends of the upper rear edge 36 of the body made of sheet material.

1. An infant garment, comprising:
(A) a body made of sheet material;
(B) left and right loop fasteners, attached to upper left and right corners of an inside surface, respectively, adjacent to an upper rear edge of a back portion of the body made of sheet material;
(C) a front loop fastener strip carried by an outer surface adjacent to an upper edge of a front portion of the body made of sheet material; and
(D) left and right fastener extensions connecting left and right ends of the front loop fastener strip to the left and right loop fasteners, respectively.

2. The infant of claim 1, additionally comprising:
(A) left and right outside rear fastening patches, having loop surfaces, carried by an outside surface of the back portion of the body made of sheet material; and
(B) left and right auxiliary fasteners attached to a middle portion of the left and right fastener extensions, respectively, wherein each auxiliary fastener includes a first surface having hook fasteners and a smooth second surface, and wherein the hook fasteners of the first surface of the left and right auxiliary fasteners are attachable to the loop surfaces of the left and right outside rear fastening patches, respectively.

3. A garment for wear by an infant, comprising:
(A) a body made of sheet material;
(B) left and right loop fasteners, attached to upper left and right corners of an inside surface, respectively, adjacent to an upper rear edge of a back portion of the body made of sheet material;
(C) a front loop fastener strip carried by an outer surface adjacent to an upper edge of a front portion of the body made of sheet material;
(D) left and right fastener extensions connecting left and right ends of the front loop fastener strip to the left and right loop fasteners, respectively;
(E) left and right outside rear fastening patches, having loop surfaces, carried by an outside surface of the back portion of the body made of sheet material; and
(F) left and right auxiliary fasteners attached to a middle portion of the left and right fastener extensions, respectively, wherein each auxiliary fastener includes a first surface having hook fasteners and a smooth second surface, and wherein the hook fasteners of the first surface of the left and right auxiliary fasteners are attachable to the loop surfaces of the left and right outside rear fastening patches, respectively.
